(19) United States
(12) Patent Application Publication
(10) Pub. No.: US 2026/0041495 A1
(43) Pub. Date: Feb. 12, 2026

(54) SURGICAL NAVIGATION SYSTEM, SURGICAL NAVIGATION METHOD, CALIBRATION METHOD OF SURGICAL NAVIGATION SYSTEM

(71) Applicant: UPSURGEON S.R.L., Assago (Milano) (IT)

(72) Inventor: Federico NICOLOSI, Assago (Milano) (IT)

(21) Appl. No.: 19/102,449

(22) PCT Filed: Aug. 10, 2023
(86) PCT No.: PCT/IB2023/058094

§ 371 Date: Feb. 10, 2025

(30) Foreign Application Priority Data
Aug. 11, 2022 (IT) 102022000017214

(51) Int. Cl. A61B 34/20; A61B 90/00; A61B 90/96; G06T 7/70

(52) U.S. Cl. CPC A61B 34/20; A61B 90/96; G06T 7/70; A61B 2034/2055; A61B 2034/2068; A61B 2034/2072; A61B 2090/3937; A61B 2560/0223; A61B 2560/0431; G06T 2207/30004; G06T 2207/30204

(57) ABSTRACT

A surgical navigation system has a mobile electronic device transportable in an operator's hand, a marker detectable by a camera of the mobile electronic device, and suitable for being placed near a portion of a human body or near a three-dimensional physical reproduction that at least partially simulates an anatomical part of the human body. A pointer device is fixed to the mobile electronic device so that the pointer device is integral in rototranslation to the mobile electronic device and a pointing end of the pointer device is visible in a field of view of the camera during a surgical navigation and/or a calibration procedure. An electronic processing unit of the mobile electronic device performs geometric operations for defining a 3D scenario reference system associated with the marker in a 3D scenario space and calculates a three-dimensional position of the pointing end in the 3D scenario reference system.

### FIELD OF APPLICATION

The present relates generally to the field of surgical navigation using three-dimensional tracking systems of surgical instruments and patients.

Operative navigation is an established technology in several surgical disciplines. It enables a real-time correspondence between a body region and a patient's radiological imaging to be carried out in order to optimize the planning and execution of a surgical procedure. In particular, when applied to neurosurgery, it is usually referred to as neuronavigation. One example of neuronavigation is in the field of brain tumor surgery: with this technology, the surgeon is able to see in real time on the patient's magnetic resonance images the site of the pathology in relation to the patient's head, and this allows planning of the surgical approach and trajectory with greater precision and reliability. In addition, intraoperatively, the navigation allows the surgeon to be guided during the surgical resection of the tumor and reduces the risk of disorientation or partial resection of the tumor.

Inconveniently, a neuronavigation system is generally a complex and expensive technology. In the case of navigation systems based on optical technology, there are infrared cameras positioned overhead and movable by means of a wheeled trolley, an infrared marker fixed integrally to the patient's head (e.g., via a Mayfield head holder), and an additional infrared marker fixed to a pointer or to each surgical instrument that needs to be tracked, a computer for data processing, and a monitor.

In a particularly disadvantageous way, in order to track a pointer so that the pointer's location may be visualized on the diagnostic images (MRI or CT), it is necessary for the pointer to have its own marker visible by the infrared cameras.

Additionally, inconveniently, traditional navigation systems are very complex and expensive, especially when they have to be used in surgical simulations for surgical training, where the simplicity of use, low cost, and accessibility to a wide audience becomes crucial for training also in underdeveloped areas.

### SOLUTION OF THE INVENTION

Thus, there is a strong need to provide a navigation system and method that overcomes the aforementioned drawbacks of systems of the prior art. In particular, a need is felt for a simpler, less expensive, and more easily accessible system and method of navigation.

Another object of the present invention is to provide a system for surgical simulation and navigation that is as realistic as possible during the navigation.

Another object of the present invention is to provide a surgical navigation device which is easily reusable in several different training sessions.

Further, an object of the present invention is to provide a surgical navigation device which is versatile in modifying the training scenario and which does not require complicated operations for modifying the surgical training scenario or for renewing it after use.

These needs are met by a surgical navigation system, a surgical navigation method, and a calibration method of a surgical navigation system according to the attached independent claims. The claims dependent thereon describe preferred or advantageous embodiments of the invention, comprising further advantageous features.

### DESCRIPTION OF THE DRAWINGS

The features and advantages of the system for surgical navigation, the surgical navigation method, and the calibration method will be evident from the description below of some preferred embodiment examples, given as an indication and without limitation, with reference to the attached figures, wherein:

FIG. 1 shows a three-dimensional perspective view of a system for surgical navigation, before the calibration step, according to an embodiment of the present invention;

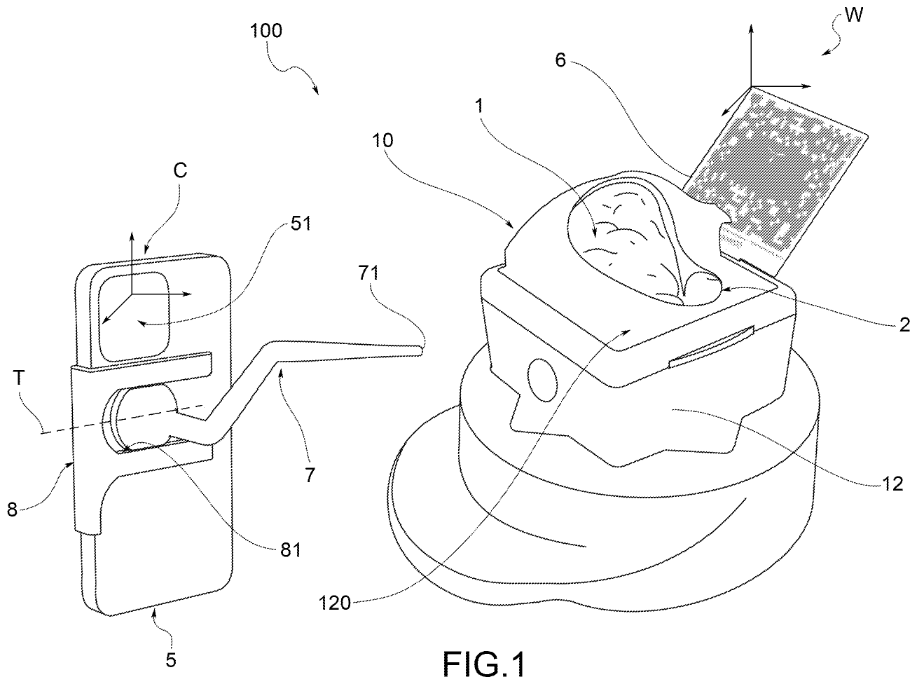

FIG. 2 shows a three-dimensional perspective view of a system for surgical navigation, during one step of the calibration method, according to an embodiment of the present invention;

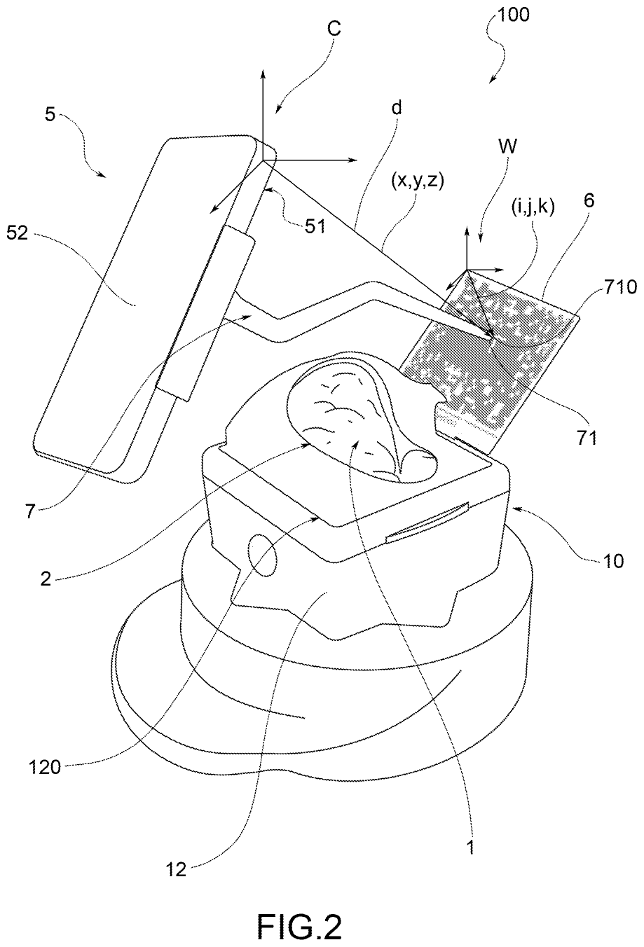

FIG. 3 shows a three-dimensional perspective view of a system for surgical navigation, during a step of surgical navigation, according to an embodiment of the present invention;

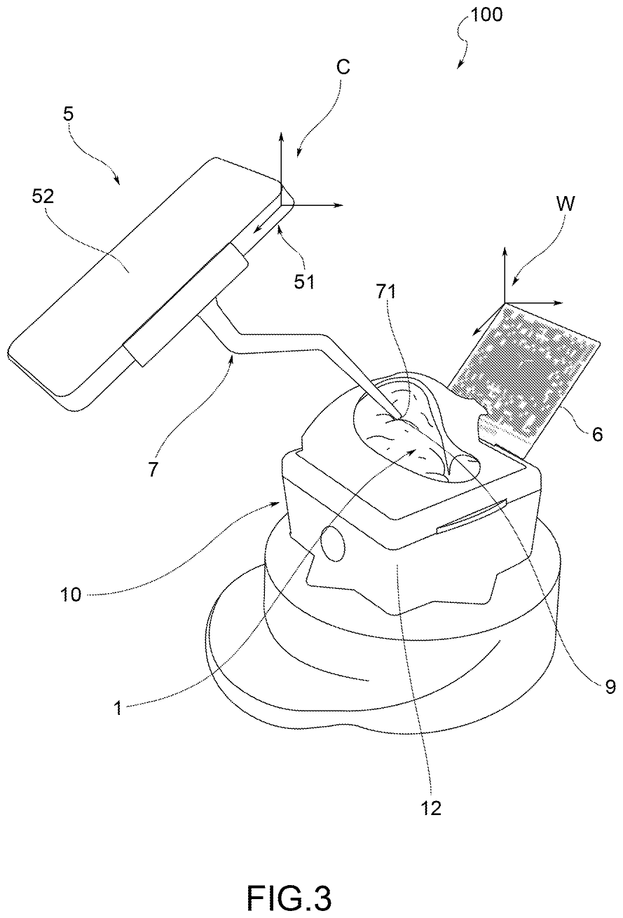

FIG. 4 shows an exploded view of a mobile electronic device, a pointer device, and a magnetic coupling device, according to an embodiment of the present invention;

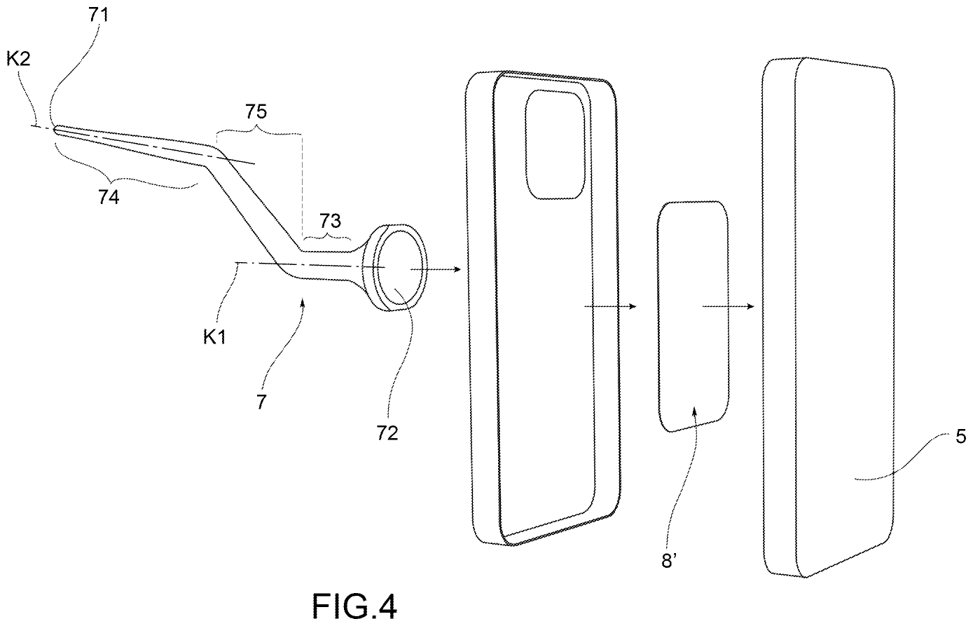

FIG. 5 shows a mobile electronic device display during two successive steps of the calibration method according to an embodiment of the present invention, for identifying the end digital image;

FIG. 6 shows a display of the mobile electronic device during one step of the surgical navigation method according to an embodiment of the present invention.

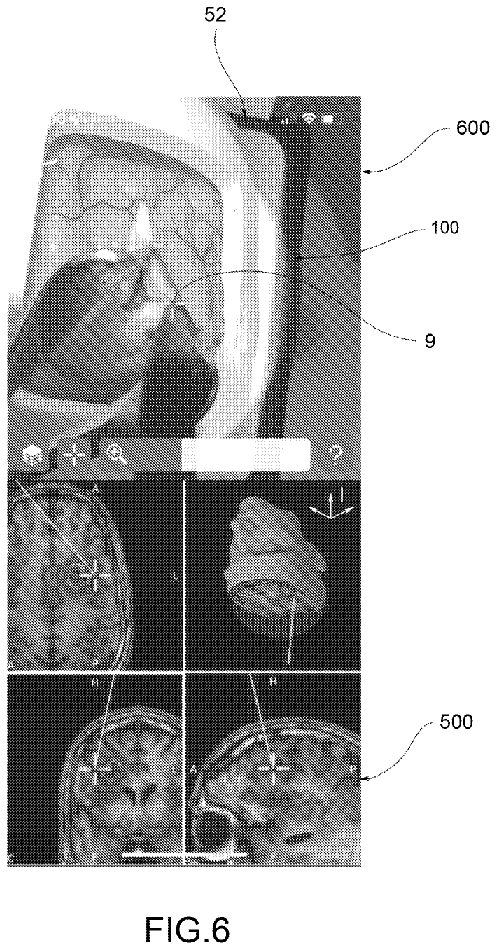

### DETAILED DESCRIPTION

With reference to the aforesaid figures, a surgical simulation device 10 is collectively indicated with the reference number 10.

The surgical simulation device 10 comprises a three-dimensional physical reproduction 1 suitable for at least partially simulating an anatomical part of the human body.

In the present discussion, the term “three-dimensional physical reproduction” means a phantom, i.e., an artificial three-dimensional reconstruction, suitable for representing an anatomical part of the human body.

It is evident that the present invention, described here for the purposes of clarity for the surgical simulation on an anatomical portion of a human body, is also suitable, with the appropriate modifications, for surgical simulation on an anatomical portion of an animal body, for example for veterinary surgical training.

In a preferred embodiment of the present invention, the three-dimensional physical reproduction 1 is suitable for simulating a portion of the human brain.

Furthermore, in an embodiment of the present invention, the three-dimensional physical reproduction 1 comprises a plurality of sub-reconstructions suitable for representing two or more anatomical elements.

Preferably the anatomical elements comprise one or more of the following: cerebral/cerebellar parenchyma, brain stem, cranial nerves, arterial/venous vessels, venous sinuses, meninges (dura mater, arachnoid mater, pia mater), skull, each of the sub-reconstructions is made with a material which reproduces the mechanical features of the corresponding real anatomical element.

In addition, in an embodiment, the surgical simulation device 10 comprises an outer frame 12 that comprises a cartridge seat 120 in which a cartridge 2 that houses the three-dimensional physical reproduction 1 is accommodated.

Preferably, the cartridge 2 is accommodated in the cartridge seat 120 in a removable manner, thus facilitating the change of surgical scenario.

With reference to the attached figures, the present invention pertains in particular to a system for surgical navigation 100. The surgical navigation system according to the present invention is suitable for use in surgical simulation for training, or for the intraoperative stage.

Such a system comprises a mobile electronic device 5 transportable in an operator's hand, such as a tablet or a smartphone. Such a mobile electronic device 5 comprises at least one electronic processing unit (e.g., one or more CPUs and/or GPUs), a display 52 and a camera 51. It is evident that the term “camera” also refers to a generic camera or in any case an image acquisition device.

The surgical navigation system 100 also comprises a marker 6 (also known in the industry as a tracker), detectable by the camera 51 of the mobile electronic device 5 and suitable for placement near a portion of the human body or near a three-dimensional physical reproduction 1, for example near the surgical simulation device 10, which at least partially simulates an anatomical part of the human body.

Preferably, the marker 6 is a depiction of a QR-code or in any case is a depiction of two-dimensional coding, e.g., a two-dimensional physical image comprising predetermined geometric features identifiable by the camera, known in the field of calibration of three-dimensional spaces for augmented reality.

The surgical navigation system 100 also comprises a pointer device 7, such as a pointing stick, having a pointing end 71.

The pointer device 7 is fixed to the mobile electronic device 5 or is releasably fixed to the mobile electronic device 5. In this way, when the pointer device 7 is fixed to the mobile electronic device 5, such a pointer device 7 is integral in rototranslation to the mobile electronic device. Further, the pointing end 71 is visible in the field of view of the camera 51 during the surgical navigation and/or during a calibration procedure.

According to one aspect of the present invention, a pointer assembly, composed of the pointer device 7 and the mobile electronic device 5, is subject matter, per se, of the present invention.

Additionally, the electronic processing unit is configured to perform geometric operations for defining a 3D scenario reference system W associated with the marker 6 in the 3D scenario space and for calculating the three-dimensional position of the pointing end 71 in this 3D scenario reference system W. Preferably, the geometric operations for defining a 3D scenario reference system W associated with the marker are operations known to the person skilled in the art, typical for camera calibration in the field of augmented reality, e.g., by means of known algorithms already implemented in available software libraries, such as ARtoolkit, ArtoolkitX, and the like. Therefore, the present discussion will not delve into these operations or the operations of linear geometry and transformations between three-dimensional spaces, as they are known to the person skilled in the art.

Therefore, preferably, the electronic processing unit is configured to perform the calculation of position and/or orientation coordinates in the 3D scenario reference system W for the pointer device 7 (and its pointing end 71) and/or the mobile electronic device 5 and/or the camera 51. In particular, the calculation of the position and/or orientation coordinates in a three-dimensional virtual or augmented reality space (3D scenario reference system) by the electronic processing unit is calculated by a technique of generating a three-dimensional virtual space and related tracking in said three-dimensional virtual space by acquiring images from a camera 51, possibly also with orientation or acceleration data obtainable from orientation and acceleration sensors on the mobile electronic device 5. Such a technique of generating three-dimensional virtual spaces is known to the person skilled in the art and experienced in virtual and augmented reality software, e.g., through known algorithms already implemented in available software libraries, such as ARtoolkit, ArtoolkitX and the like.

As is clearly appreciable from this description, when the pointer device 7 is fixed to the mobile electronic device 5, its pointing end 71 is visible in the field of view of the camera 51; therefore, the pointer device 7 and the camera 51 are preferably tracked in the 3D scenario space W only due to a calculation of their spatial coordinates performed by the electronic processing unit, which is configured to perform geometric operations for the definition of a 3D scenario reference system W associated with the marker 6 in the 3D scenario space. Therefore, the pointer device 7 and the camera 51 are not tracked by another external tracking device or system, e.g., they are also not tracked by an additional infrared optoelectronic or electromagnetic tracking system known in the art that uses optical reflection markers or electromagnetic markers (e.g., coils). This makes it possible to radically simplify the entire system for surgical navigation 100. Preferably, the mobile electronic device 5 is also tracked in the 3D scenario space 3 only by a calculation of its spatial coordinates performed by the electronic processing unit that processes the images of the camera 51 and the image of the marker 6 and is not tracked by another tracking device or system external to said mobile electronic device 5.

According to an embodiment, as mentioned above, the system for surgical navigation 100 comprises a three-dimensional physical reproduction 1 suitable for simulating at least partially an anatomical part of the human body, or an anatomical portion of a human body, such as a skull. In this case, the marker 6 is removably fixed in close proximity to the three-dimensional physical reproduction 1, as shown in the attached figures, or to the anatomical portion of a human body (e.g., attached to a Mayfield head clamp).

According to an embodiment, the system 100 comprises a pointer coupling device 8 suitable for coupling to the mobile electronic device 5 and comprising a coupling seat 81 shaped to accommodate a rear end 72 of the pointer device 7 opposite the pointing end 71.

Preferably, the coupling seat 81 is shaped so as to couple in a form-fit with the rear end 72.

Preferably, the rear end 72 is shaped to be accommodated in the coupling seat 81 translatably along a pointer slide direction T and to remain fixed in the coupling seat 81 once it has reached a stationary position in said coupling seat 81.

According to a variant embodiment, the system 100 comprises a magnetic pointer coupling device 8′, comprising a magnetic or ferromagnetic material, and suitable for being joined to the mobile electronic device 5. In this variant, the pointer device 7 comprises a rear end 72 of the pointer device 7, opposite the pointing end 71 and provided with a magnetic or ferromagnetic material for magnetic coupling with the magnetic pointer coupling device 8′.

Preferably, the pointing device 7 is a pointing stick, extending predominantly between the pointing end 71 and a rear end 72 arranged on the opposite side from the pointing end 71. This pointing stick comprises:
a proximal portion 73, arranged near the rear end 72, and extending predominantly along a first longitudinal direction K1;
a distal portion 74, comprising the pointing end 71, and extending predominantly along a second longitudinal direction K2, spaced from, and preferably parallel to, the first longitudinal direction K1;
a connecting portion 75 that connects the proximal portion 73 with the distal portion 74.

The aforesaid configuration of the pointing stick allows the stick to be fixed above or below the camera 51, while at the same time ensuring adequate visibility of the pointing end 71 in the camera 51.

Preferably, the pointing stick is shaped according to a sigmoidal or “S” or “Z” shape.

As mentioned, the present invention pertains to a method of surgical navigation.

It should be remembered that, in the present discussion, when reference is made to the term “position,” it will refer to a position in the most general sense of the geometric term, i.e., it will refer both to the Cartesian position with respect to the chosen reference system and to the rotation or rototranslation matrix, if any, that defines the position of an object in space, unless it is punctiform object. In other words, for example, when referring to the position of one reference system with respect to another reference system, both the translation and rotation of that reference system with respect to the other reference system (i.e., the relative rototranslation between the two systems) are intended.

It is also evident that the term “surgical navigation method” is not to be understood as a method of surgical treatment, but rather, as already explained in the introduction of this document, as a method for navigating instruments to track on the patient the anatomical structures visualized on the radiographic examinations, e.g. computerized tomography and magnetic resonance imaging. By using these examinations as a map and the instruments as probes, the navigator enables one to know in real time where the instruments or the healthy and pathological anatomical structures are located.

For example, as will be evident from the remainder of the present description, the surgical navigation method may be used on mock anatomical models of the human body, such as a three-dimensional physical reproduction 1, and is therefore not used for surgical treatment of a living human or animal body. Furthermore, even if the surgical navigation method were performed on a human body or an anatomical portion of a human body, it is still not to be considered a method of surgical treatment because no step that will be described with reference to the surgical navigation method entails injury to the human or animal body to which it is applied.

The method of surgical navigation according to the present invention comprises at least the following operational steps:
i) providing a surgical navigation system 100 as described in one of the embodiments of the present discussion;
ii) providing digital images 500 related to a virtual digital representation of the three-dimensional physical reproduction 1 or to a virtual digital representation of the anatomical portion of a human body or part thereof, for example magnetic resonance imaging (MRI) or computed tomography (CT) images; it is evident that, as known in the field, such digital images 500 are positioned in a virtual image space, with respect to a virtual reference system I;
iii) framing a region of the three-dimensional physical reproduction 1, or a region of the anatomical portion of a human body, with the camera 51;
iv) simultaneously with step iii), framing the pointing end 71 with the camera 51;
v) by moving the mobile electronic device 5, causing the movement of the pointing end 71 and bringing the pointing end 71 closer to a physical point 9 of the three-dimensional physical reproduction 1 or the anatomical portion of a human body;
vi) on the electronic processing unit:
aa) calculating a virtual three-dimensional position of the pointing end 71 when located at the physical point 9 with respect to the virtual reference system I, i.e., in the virtual image space;
bb) selecting one or more digital image(s) of said digital images 500, positioned at the spatial coordinates of the virtual three-dimensional position of the pointing end 71, for the display thereof.

Preferably, after step vi), i.e., after selecting the digital images at the spatial coordinates of the virtual three-dimensional position of the pointing end 71, the method comprises step vii) of displaying on the display 52 of the mobile electronic device 5 the one or more digital images selected in said step vi). In this way, the operator is able to perform true surgical navigation, as diagnostic images corresponding to the current position of the pointer device 71 in the 3D scenario space W are presented instant by instant and position by position.

According to one embodiment of the method, at the same time as step vii), a current image 600 of the three-dimensional physical reproduction 1 or anatomical portion of a human body, or part thereof, captured by the camera 51 is also shown on the display 52.

The present invention also pertains to a calibration method of a system for surgical navigation 100 described in the present discussion. Such a calibration method comprises the steps of:
a) providing the pointer device 7 fixed to the mobile electronic device 5 so that the pointing end 71 is visible in the field of view of the camera 51 and integral in motion therewith;
b) by means of the camera 51, acquiring one or more images of the marker 6 and, on the electronic processing unit, constructing a three-dimensional scenario space in a 3D scenario reference system W and calculating a position of a 3D camera reference system C, integral with camera 51, in said 3D scenario reference system W;
c) by means of the camera 51, acquiring a first 2D pointer image 600 that contains the end digital image 71′ of the pointing end 71, i.e., an end digital point; d) identifying two end coordinates (x, y) of the end digital image 71′ and storing said two end coordinates x, y with respect to the 3D camera reference system C, on a storage device of the mobile electronic device 5; preferably the two end coordinates are calculated in pixels;
e) positioning the pointing end 71 in contact with a physical calibration point 710, this physical calibration point 710 being a physical point at known three-dimensional coordinates (i, j, k) in the 3D scenario reference system W;
f) acquiring the position of the 3D camera reference system C in the 3D scenario reference system W;
g) on the electronic processing unit, calculating a geometric distance d between the 3D camera reference system C and the physical calibration point 710 in the 3D camera reference system C and, as a function of said geometric distance d, calculating a third end coordinate z, which, together with the two end coordinates x, y defines the position of the pointing end 71 with respect to the 3D camera reference system C.

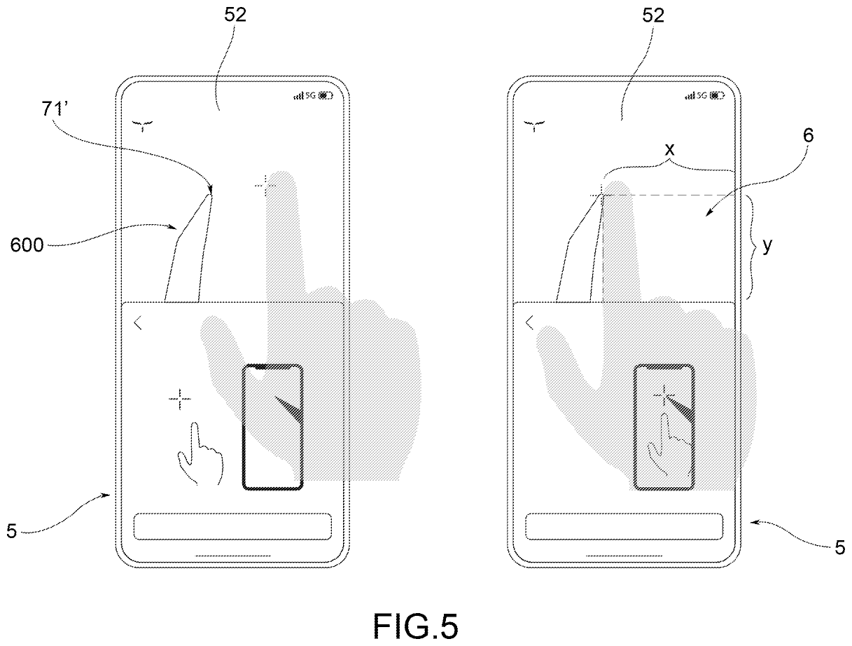

Preferably, step c) of identifying two end coordinates x, y of the end digital image 71′ comprises the steps of displaying said first 2D pointer image on the display 52 (as for example shown in FIG. 5) and on said first 2D pointer image manually selecting, by an operator, the end digital image 71′, for example by pressing on the touch display at the exact point of the end digital image 71′, so that the electronic processing unit may calculate and save the coordinates of the point pressed by the operator on the display 52.

According to variant, step c) of identifying two end coordinates (x, y) of the end digital image 71′ comprises the step of processing this first 2D pointer image by an image processing algorithm for the automatic extraction of the end digital image 71′, such as an image contour extraction algorithm (e.g., an edge detection algorithm) or a mask correlation algorithm.

It is evident that, preferably, the physical calibration point 710 has known three-dimensional coordinates because it is already precalibrated in the 3D scenario reference system W, for example, because it is a point belonging to the marker 6 or with a predefined geometric relationship to the marker 6.

Preferably, it is also evident that the calculation of the third end coordinate z as a function of said geometric distance d, may be calculated by application of a three-dimensional offset vector to the position of the 3D camera reference system C. Such a three-dimensional offset vector is calculated geometrically based on the geometric distance d, for each spatial coordinate.

According to an embodiment, after performing steps a) to g) of the calibration method described above, for the calculation of the virtual three-dimensional position of the pointing end 71 in step aa), the following operational steps are performed:
converting the position of the pointing end 71 from the 3D camera reference system C into the 3D scenario reference system W; this may be done since the position of the 3D camera reference system C in the 3D scenario reference system W is known, thus obtaining the position of the pointing end 71 in the 3D scenario reference system W;
converting the position of the pointing end 71 from the 3D scenario reference system W to the virtual reference system I, i.e., in the virtual image space, the virtual reference system I with the 3D scenario reference system W having been pre-registered by a registration technique between spaces, e.g., by means of a corresponding point registration technique or by image morphing registration technique, known to the person skilled in the art.

Innovatively, the present innovation successfully overcomes the drawbacks associated with the navigation systems of the prior art. In particular, the present invention makes it possible to condense all the many elements of a normal navigation system (infrared chambers, marker at the pointer, marker at the patient's head, computer, and monitor) within a single mobile electronic device, such as a smartphone or tablet, suitably integrated with a small piece of hardware that acts as a pointer device and is fixed thereto.

Additionally, the use of augmented reality technology with graphic marker recognition instead of infrared technology further simplifies the system.

Advantageously, the invention makes it possible to match what was previously delegated to infrared cameras to a camera on the mobile electronic device, which directly frames both the pointer device, appropriately positioned so as to be visible to the smartphone camera, and the surgical operating field and/or the detail to be explored with the pointer. The invention also allows the marker generally fixed to the patient (or simulator device) to match the augmented reality marker on said simulator. All this makes it possible to eliminate the need for a specific marker fixed to the pointer device since the same is already directly displayed by the camera and has a known position that corresponds to the position of the smartphone itself with respect to the augmented reality marker of the simulator itself.

Consequently, advantageously, since an appropriate spatial registration is made between the physical model (portion of the human body or simulated three-dimensional physical reproduction) and the virtual model (i.e., a virtual three-dimensional model obtained by the tomographic diagnostic images, e.g., MRI or CT), the present invention forms a true navigation system in which the full 3D tomography is made to correspond spatially to the anatomical models represented on the physical simulator or the portion of the human body, of which the tomography is in fact a graphical representation. This positioning is done by recognition by the camera of the augmented reality marker that has a pre-registered position relative to the physical simulator or portion of the human body.

Once the aforesaid steps are defined, the system according to the present invention allows the surgical operator to track the tip of the pointer device, which is automatically matched and displayed as a moving point on the axial, sagittal, and coronal images of the virtual reference system.

Advantageously, moreover, the system according to the present invention does not require careful and precise positioning of the pointer device on the mobile electronic device, since the calibration may be carried out from time to time quickly and easily by the described calibration method.

It is clear that, to the embodiments of the aforesaid invention, a person skilled in the art, in order to meet specific needs, could make variations or substitutions of elements with functionally equivalent ones. These variants are also contained within the scope of protection as defined by the following claims. Moreover, each variant described as belonging to a possible embodiment may be implemented independently of the other variants described.

1. A surgical navigation system comprising:
a mobile electronic device transportable in an operator's hand, optionally the mobile electronic device being a tablet or a smartphone, the mobile electronic device comprising at least one electronic processing unit, a display and a camera;
a marker optionally the marker being a depiction of a QR-code, detectable by the camera of the mobile electronic device and suitable for being positioned near a portion of a human body or near a three-dimensional physical reproduction which at least partially simulates an anatomical part of the human body; and
a pointer device having a pointing end,
wherein said the pointer device is fixed to the mobile electronic device or is releasably fixed to the mobile electronic device, so that, when the pointer device is fixed to the mobile electronic device the pointer device is integral in rototranslation to the mobile electronic device and said the pointing end is visible in a field of view of the camera during a surgical navigation and/or during a calibration procedure,
and wherein the electronic processing unit is configured to perform geometric operations for defining a 3D scenario reference system associated with the marker in a 3D scenario space and for calculating a three-dimensional position of the pointing end in said the 3D scenario reference system.

2. The surgical navigation system of claim 1, further comprising
a three-dimensional physical reproduction suitable for at least partially simulating an anatomical part of the human body,
or an anatomical portion of the human body, optionally the anatomical portion of the human body being a skull,
wherein the marker is removably fixed near the three-dimensional physical reproduction or near the anatomical portion of the human body.

3. The surgical navigation system of claim 1, further comprising a pointer coupling device suitable for coupling to the mobile electronic device and comprising a coupling seat shaped to receive a rear end of the pointer device opposite the pointing end.

4. The surgical navigation system of claim 1, further comprising a magnetic pointer coupling device comprising a magnetic or ferromagnetic material and suitable for being joined to the mobile electronic device, wherein the pointer device comprises a rear end, opposite the pointing end and provided with a magnetic or ferromagnetic material for magnetic coupling with the magnetic pointer coupling device.

5. The surgical navigation system of claim 1, wherein the pointer device is a pointing stick, mainly extending between the pointing end and a rear end arranged on an opposite side with respect to the pointing end, and
wherein the pointing stick comprises:
a proximal portion mainly extending along a first longitudinal direction and arranged near the rear end;
a distal portion, comprising the pointing end and mainly extending along a second longitudinal direction spaced, and preferably parallel, with respect to the first longitudinal direction; and
a connecting portion connecting the proximal portion with the distal portion.

6. The surgical navigation system of claim 5, wherein the pointing stick has a sigmoidal or “S” or “Z” shape.

7. A surgical navigation method comprising the following operating steps:
i) providing a surgical navigation system comprising:
a mobile electronic device transportable in an operator's hand, optionally the mobile electronic device being a tablet or a smartphone, the mobile electronic device comprising at least one electronic processing unit, a display and a camera;
a marker, optionally the marker being a depiction of a QR-code, detectable by the camera of the mobile electronic device and suitable for being positioned near a portion of a human body or near a three-dimensional physical reproduction which at least partially simulates an anatomical part of the human body; and
a pointer device having a pointing end,
wherein the pointer device is fixed to the mobile electronic device or is releasably fixed to the mobile electronic device, so that, when the pointer device is fixed to the mobile electronic device, the pointer device is integral in rototranslation to the mobile electronic device and the pointing end is visible in a field of view of the camera during a surgical navigation and/or during a calibration procedure,
wherein the electronic processing unit is configured to perform geometric operations for defining a 3D scenario reference system associated with the marker in a 3D scenario space and for calculating a three-dimensional position of the pointing end in the 3D scenario reference system, wherein the surgical navigation system further comprises
a three-dimensional physical reproduction suitable for at least partially simulating an anatomical part of the human body,
or an anatomical portion of the human body, optionally the anatomical portion of the human body being a skull, and
wherein the marker is removably fixed near the three-dimensional physical reproduction or near the anatomical portion of the human body;
ii) providing digital images related to a virtual digital representation of the three-dimensional physical reproduction or of the anatomical portion of the human body or part thereof, optionally the digital images being magnetic resonance (MRI) images or computed tomography (CT) images, the digital images being positioned in a virtual image space, with respect to a virtual reference system;
iii) framing a region of the three-dimensional physical reproduction, or of the anatomical portion of the human body, with the camera
iv) simultaneously with step iii), framing the pointing end with the camera;
v) by a movement of the mobile electronic device, causing a movement of the pointing end and bringing the pointing end closer to a physical point (9) of the three-dimensional physical reproduction or of the anatomical portion of the human body; and
vi) on the electronic processing unit:
aa) calculating a virtual three-dimensional position of the pointing end when located in the physical point with respect to the virtual reference system, that is to say, in the virtual image space; and
bb) selecting one or more digital images of the digital images, positioned at spatial coordinates of the virtual three-dimensional position of the pointing end, for a display thereof.

8. The surgical navigation method according to of claim 7, comprising the following operating step:
vii) after step vi), displaying the one or more digital images selected in step vi) on the display of the mobile electronic device.

9. The surgical navigation method of claim 8, wherein, simultaneously with step vii), a current image of the three-dimensional physical reproduction or of the anatomical portion of the human body, or a part thereof, captured by the camera is also displayed on the display.

10. A calibration method of a surgical navigation system comprising:
a mobile electronic device transportable in an operator's hand, optionally the mobile electronic device being a tablet or a smartphone, the mobile electronic device comprising at least one electronic processing unit, a display and a camera;
a marker, optionally the marker being a depiction of a QR-code, detectable by the camera of the mobile electronic device and suitable for being positioned near a portion of a human body or near a three-dimensional physical reproduction which at least partially simulates an anatomical part of the human body; and
a pointer device having a pointing end,
wherein the pointer device is fixed to the mobile electronic device or is releasably fixed to the mobile electronic device, so that, when the pointer device is fixed to the mobile electronic device, the pointer device is integral in rototranslation to the mobile electronic device and the pointing end is visible in a field of view of the camera during a surgical navigation and/or during a calibration procedure,
and wherein the electronic processing unit is configured to perform geometric operations for defining a 3D scenario reference system associated with the marker in a 3D scenario space and for calculating a three-dimensional position of the pointing end in the 3D scenario reference system,
the calibration method comprising the following operating steps:
a) providing the pointer device fixed to the mobile electronic device so that the pointing end is visible in the field of view of the camera and integral in motion therewith;
b) by the camera, acquiring one or more images of the marker and, on the electronic processing unit, constructing the 3D scenario space in the 3D scenario reference system and calculating a position of a 3D camera reference system, integral with the camera, in the 3D scenario reference system
c) by the camera, acquiring a first 2D pointer image containing an end digital image of the pointing end, that is to say, an end digital point;
d) identifying two end coordinates, preferably in pixels, of the end digital image and storing the two end coordinates with respect to the 3D camera reference system, on a storage device of the mobile electronic device;
e) positioning the pointing end in contact with a physical calibration point, the physical calibration point being a physical point with known three-dimensional coordinates in the 3D scenario reference system;
f) acquiring the position of the 3D camera reference system in the 3D scenario reference system; and
g) on the electronic processing unit, calculating a geometric distance between the 3D camera reference system and the physical calibration point in the 3D camera reference system and, as a function of said the geometric distance, calculating a third end coordinate, which together with the two end coordinates defines the position of the pointing end with respect to the 3D camera reference system.

11. The surgical navigation method of claim 7, wherein, after performing steps a) to g) of a calibration method of a surgical navigation system comprising:
a mobile electronic device transportable in an operator's hand, optionally the mobile electronic device being a tablet or a smartphone, the mobile electronic device comprising at least one electronic processing unit, a display and a camera;
a marker, optionally the marker being a depiction of a QR-code, detectable by the camera of the mobile electronic device and suitable for being positioned near a portion of a human body or near a three-dimensional physical reproduction which at least partially simulates an anatomical part of the human body; and
a pointer device having a pointing end,
wherein the pointer device is fixed to the mobile electronic device or is releasably fixed to the mobile electronic device, so that, when the pointer device is fixed to the mobile electronic device, the pointer device is integral in rototranslation to the mobile electronic device and the pointing end is visible in a field of view of the camera during a surgical navigation and/or during a calibration procedure,
and wherein the electronic processing unit is configured to perform geometric operations for defining a 3D scenario reference system associated with the marker in a 3D scenario space and for calculating a three-dimensional position of the pointing end in the 3D scenario reference system,
the calibration method comprising the following operating steps:
a) providing the pointer device fixed to the mobile electronic device so that the pointing end is visible in the field of view of the camera and integral in motion therewith;
b) by the camera, acquiring one or more images of the marker and, on the electronic processing unit, constructing the 3D scenario space in the 3D scenario reference system and calculating a position of a 3D camera reference system, integral with the camera, in the 3D scenario reference system;
c) by the camera, acquiring a first 2D pointer image containing an end digital image of the pointing end, that is to say, an end digital point;
d) identifying two end coordinates, preferably in pixels, of the end digital image and storing the two end coordinates with respect to the 3D camera reference system, on a storage device of the mobile electronic device;
e) positioning the pointing end in contact with a physical calibration point, the physical calibration point being a physical point with known three-dimensional coordinates in the 3D scenario reference system;
f) acquiring the position of the 3D camera reference system the 3D scenario reference system; and
g) on the electronic processing unit, calculating a geometric distance between the 3D camera reference system and the physical calibration point in the 3D camera reference system and, as a function of the geometric distance, calculating a third end coordinate, which together with the two end coordinates defines the position of the pointing end with respect to the 3D camera reference system,
the surgical navigation method comprises the following operating steps are comprised for calculating the virtual three-dimensional position of the pointing end in step aa):
converting the position of the pointing end from the 3D camera reference system to the 3D scenario reference system the position of the 3D camera reference system in the 3D scenario reference system being known, obtaining the position of the pointing end in the 3D scenario reference system; and
converting the position of the pointing end from the 3D scenario reference system to the virtual reference system, that is to say, in the virtual image space, the virtual reference system having been pre-registered with the 3D scenario reference system by a space registration technique, optionally by corresponding point registration technique or by image morphing registration technique.
